(19) United States
(12) Patent Application Publication
(10) Pub. No.: US 2024/0308977 A1
(43) Pub. Date: Sep. 19, 2024

(54) PYRIDAZINE DERIVATIVES AS INHIBITORS OF NLRP3

(71) Applicant: Hoffmann-La Roche Inc., Little Falls, NJ (US)

(72) Inventors: Lewis Scott AITKEN, Nottingham (GB); Thomas Alexander ALANINE, Nottingham (GB); Lea Aurelie BOUCHE, Basel (CH); Wolfgang GUBA, Muellheim (DE); Georg JAESCHKE, Basel (CH); Stefanie Katharina MESCH, Basel (CH); Stephen Malcom THOM, Nottingham (GB); Andreas Michael TOSSTORFF, Muenchenstein (CH); Sabrina HERR, Horgen (CH); Christian SCHNIDER, Biel-Benken (CH); Sandra STEINER, Sursee (CH)

(73) Assignee: Hoffmann-La Roche Inc., Little Falls, NJ (US)

(21) Appl. No.: 18/666,566

(22) Filed: May 16, 2024

Related U.S. Application Data
(63) Continuation of application No. PCT/EP2022/082174, filed on Nov. 17, 2022.

(30) Foreign Application Priority Data
Nov. 19, 2021 (EP) 21209168.0
Aug. 2, 2022 (EP) 22188185.7

(51) Int. Cl. C07D 401/12; A61K 31/501; C07D 237/20; C07D 471/04

(52) U.S. Cl. CPC C07D 401/12; A61K 31/501; C07D 237/20; C07D 471/04

(57) ABSTRACT

The invention relates to novel compounds having the general formula Ib

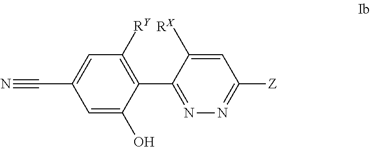

wherein R^{X}, R^{Y} and Z is described herein, composition including the compounds and methods of using the compounds.

### FIELD OF THE INVENTION

The present invention relates to organic compounds useful for therapy and/or prophylaxis in a mammal, and in particular to compounds that modulate NLRP3 inhibition.

The present invention provides novel compounds of formula Ib,

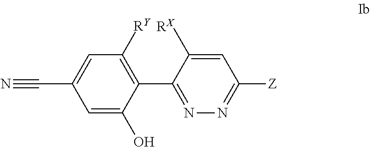

wherein
R^{X} is selected from H, alkyl and haloalkyl and R^{Y} is selected from H, alkyl, alkoxyalkyl and halo, provided that if R^{X} is H then R^{Y} is not H;
Z is selected from systems R, S, and T

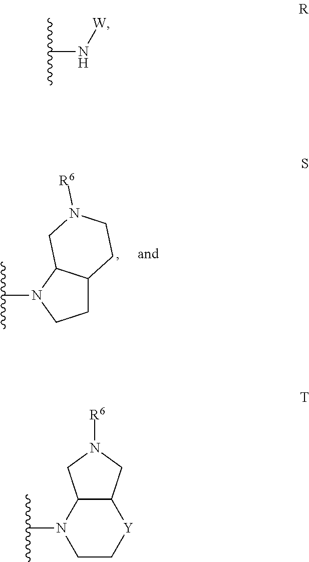

wherein systems S and T can be further substituted by OH, halo, alkyl or cyano;
R⁶ is H or alkyl;
Y is CH₂, O or NR⁷;
R⁷ is H or alkyl;
W is a substituted 4-6-membered-cycloalkyl ring or a substituted heterocycle of ring system A, wherein substituted cycloalkyl is substituted with 1 or 2 substituents selected from OH, halo, and alkyl, and ring system A is

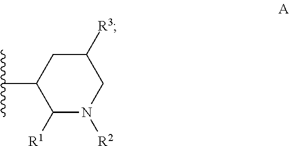

R¹ is H and R² is alkyl, or R¹ and R², and the atoms to which they are bonded, together form a 5-member ring optionally substituted with OH or halo;
R³ is H, OH or halo;
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1B and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1B and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula Ib,

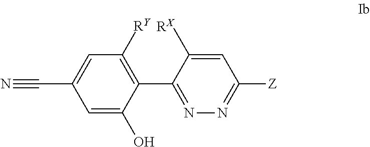

wherein
R^{X} is selected from H, alkyl and haloalkyl and R^{Y} is selected from H, alkyl, alkoxyalkyl and halo, provided that if R^{X} is H then R^{Y} is not H;
Z is selected from systems R, S, and T

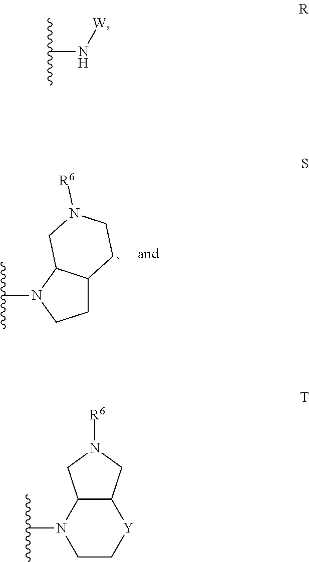

wherein systems S and T can be further substituted by OH, halo, alkyl or cyano;
R⁶ is H or alkyl;
Y is CH₂, O or NR⁷;
R⁷ is H or alkyl;
W is a substituted 4-6-membered-cycloalkyl ring or a substituted heterocycle of ring system A, wherein substituted cycloalkyl is substituted with 1 or 2 substituents selected from OH, halo, and alkyl, and ring system A is

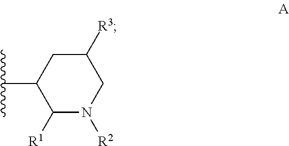

R¹ is H and R² is alkyl, or R¹ and R², and the atoms to which they are bonded, together form a 5-member ring optionally substituted with OH or halo;
R³ is H, OH or halo;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl.

The term “alkoxy” denotes a group of the formula-O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy.

The term “alkoxyalkyl” denotes a C₁₋₆-alkyl group wherein at least one of the hydrogen atoms of the C₁₋₆-alkyl group is replaced by a C₁₋₆-alkoxy group.

The term “cyano” denotes a —C≡N group.

The term “halogen”, “halide” and “halo” are used interchangeably herein and denote fluoro, chloro, bromo or iodo. Particular halo is fluoro.

The term “haloalkyl” denotes a C₁₋₆-alkyl group wherein at least one of the hydrogen atoms of the C₁₋₆-alkyl group has been replaced by the same or different halogen atoms. Particular example of haloalkyl is trifluoromethyl.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl, cyclohexyl, octahydropentalenyl, spiro[3.3]heptanyl, and the like. Particular examples include cyclobutyl.

The term “heterocycle” denotes a monovalent saturated or partly unsaturated mono- or bicyclic ring system of 4 to 10 ring atoms, or 4 to 9 ring atoms, comprising 1, 2, or 3 ring heteroatoms selected from N, O and S, the remaining ring atoms being carbon. Examples for monocyclic saturated heterocycle rings are oxetanyl, azetidinyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, or piperazinyl. Examples for partly unsaturated heterocycle rings are dihydrofuryl, imidazolinyl, dihydro-oxazolyl, tetrahydro-pyridinyl, or dihydropyranyl. Particular example of a heterocycle ring is piperidyl.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol uM.

The abbreviation uL means microliter and is equivalent to the symbol uL.

The abbreviation ug means microgram and is equivalent to the symbol ug.

The compounds of formula Ib can contain one or several asymmetric centers and can be present in the form of optically pure enantiomers, mixtures of enantiomers such as, for example, racemates, optically pure diastereoisomers, mixtures of diastereoisomers.

The compounds of formula I can contain one or several asymmetric centers and can be present in the form of optically pure enantiomers, mixtures of enantiomers such as, for example, racemates, optically pure diastereoisomers, mixtures of diastereoisomers.

According to the Cahn-Ingold-Prelog Convention the asymmetric carbon atom can be of the “R” or “S” configuration.

Also an embodiment of the present invention provides compounds according to formula Ib as described herein and pharmaceutically acceptable salts or esters thereof, in particular compounds according to formula I as described herein and pharmaceutically acceptable salts thereof, more particularly compounds according to formula Ib as described herein.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds of formula Ib as described herein, wherein RN is H or alkyl.

An embodiment of the present invention provides compounds of formula Ib as described herein, wherein R^{Y} is H or alkyl.

An embodiment of the present invention provides compounds of formula Ib as described herein, wherein R^{Y} is selected from H, alkyl and haloalkyl and R^{Y} is selected from H or alkyl, provided that if R^{X} is H then R^{Y} is not H;

An embodiment of the present invention provides compounds of formula Ib as described herein, wherein R^{X} is selected from H or alkyl and R^{Y} is selected from H or alkyl, provided that if R^{X} is H then R^{Y} is not H;

An embodiment of the present invention provides compounds of formula Ib as described herein, wherein Z is system R or system S.

An embodiment of the present invention provides compounds of formula Ib as described herein, wherein Z is system S.

An embodiment of the present invention provides compounds of formula Ib as described herein, wherein R⁶ is alkyl.

An embodiment of the present invention provides compounds of formula Ib as described herein, wherein W is a 4-membered cycloalkyl ring substituted with alkyl and OH, or W is a substituted heterocycle of ring system A.

An embodiment of the present invention provides compounds of formula Ib as described herein, wherein W is a 4-membered cycloalkyl ring substituted with alkyl and OH, or W is a substituted heterocycle of ring system A wherein R¹ is H, R² is alkyl and R³ is H.

An embodiment of the present invention provides compounds of formula Ib as described herein, wherein W is a substituted heterocycle of ring system A.

An embodiment of the present invention provides compounds of formula Ib as described herein, wherein R¹ is H, R² is alkyl and R³ is H or alkyl.

An embodiment of the present invention provides compounds of formula Ib as described herein, wherein R¹ is H, R² is alkyl and R³ is H.

An embodiment of the present invention provides compounds of formula Ib as described herein, wherein
R^{X} is selected from H, alkyl and haloalkyl and R^{Y} is selected from H and alkyl, provided that if R^{X} is H then R^{Y} is not H;
Z is selected from systems R and S

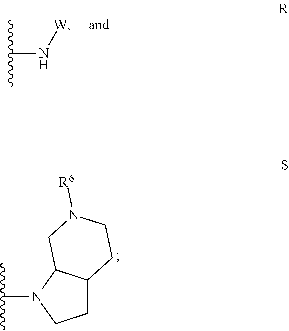

R⁶ is alkyl;
W is a substituted 4 membered-cycloalkyl ring substituted with alkyl and OH, or W is a substituted heterocycle of ring system A,

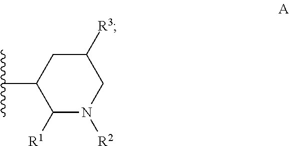

R¹ is H and R² is alkyl;
R³ is H;
and pharmaceutically acceptable salts thereof.

An embodiment of the present invention provides compounds of formula Ib as described herein, wherein
R^{X} is selected from H or alkyl and R^{Y} is selected from H and alkyl, provided that if R^{X} is H then R^{Y} is not H;
Z is selected from systems R and S

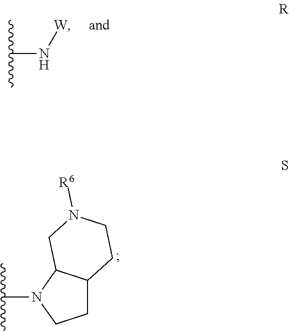

R⁶ is alkyl;
W is a substituted heterocycle of ring system A,

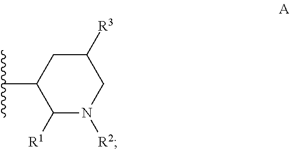

R¹ is H and R² is alkyl;
R³ is H;
and pharmaceutically acceptable salts thereof.

An embodiment of the present invention provides compounds of formula Ib as described herein, wherein
R^{X} is selected from H and alkyl and R^{Y} is selected from H and alkyl, provided that if R^{X} is H then R^{Y} is not H;
Z is system S

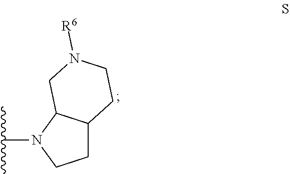

R⁶ is alkyl;
and pharmaceutically acceptable salts thereof.

Particular examples of compounds of formula Ib as described herein are selected from
4-[6-[[(3R)-1-Ethyl-3-piperidyl]amino]pyridazin-3-yl]-3-hydroxy-5-methyl-benzonitrile;
4-[6-[[(3R)-1-Ethyl-3-piperidyl]amino]-4-(trifluoromethyl)pyridazin-3-yl]-3-hydroxy-benzonitrile;
3-Hydroxy-4-[6-[(3-hydroxy-3-methyl-cyclobutyl)amino]-4-methyl-pyridazin-3-yl]benzonitrile;
4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aS,7aR)-6-Methyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aR,7aS)-6-methyl-3,3a,4,5,7,7a-hexahydro-2H-pyrrolo[2,3-c]pyridin-1-yl]pyridazin-3-yl]-3-hydroxy-5-methyl-benzonitrile;
   and pharmaceutically acceptable salts thereof.

Also particular examples of compounds of formula Ib as described herein are selected from
4-[6-[(3aS,7aR)-6-Ethyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aR,7aS)-6-ethyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aR,7aS)-6-Ethyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-ethyl-3,3a,4,5,7,7a-hexahydro-2H-pyrrolo[2,3-c]pyridin-1-yl]pyridazin-3-yl]-3-hydroxy-5-methyl-benzonitrile;
3-Hydroxy-4-[4-methyl-6-(6-methyl-3,3a,4,5,7,7a-hexahydro-2H-pyrrolo[2,3-c]pyridin-1-yl)pyridazin-3-yl]benzonitrile;
4-[6-[(3aS,7aR)-6-Methyl-3,3a,4,5,7,7a-hexahydro-2H-pyrrolo[2,3-c]pyridin-1-yl]-4-methyl-pyridazin-3-yl]-3-hydroxy-benzonitrile or 4-[6-[(3aR,7aS)-6-methyl-3,3a,4,5,7,7a-hexahydro-2H-pyrrolo[2,3-c]pyridin-1-yl]-4-methyl-pyridazin-3-yl]-3-hydroxy-benzonitrile;
4-[6-[(3aR,7aS)-6-Methyl-3,3a,4,5,7,7a-hexahydro-2H-pyrrolo[2,3-c]pyridin-1-yl]-4-methyl-pyridazin-3-yl]-3-hydroxy-benzonitrile or 4-[6-[(3aS,7aR)-6-methyl-3,3a,4,5,7,7a-hexahydro-2H-pyrrolo[2,3-c]pyridin-1-yl]-4-methyl-pyridazin-3-yl]-3-hydroxy-benzonitrile;
4-[4-Ethyl-6-[[(3R)-1-ethyl-3-piperidyl]amino]pyridazin-3-yl]-3-hydroxy-benzonitrile;
and pharmaceutically acceptable salts thereof.

Preferred examples of compounds of formula Ib as described herein are selected from
4-[6-[[(3R)-1-Ethyl-3-piperidyl]amino]pyridazin-3-yl]-3-hydroxy-5-methyl-benzonitrile;
4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aS,7aR)-6-Methyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aR,7aS)-6-methyl-3,3a,4,5,7,7a-hexahydro-2H-pyrrolo[2,3-c]pyridin-1-yl]pyridazin-3-yl]-3-hydroxy-5-methyl-benzonitrile;
and pharmaceutically acceptable salts thereof.

Also preferred examples of compounds of formula Ib as described herein are selected from
4-[6-[(3aS,7aR)-6-Ethyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aR,7aS)-6-ethyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aR,7aS)-6-Ethyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-ethyl-3,3a,4,5,7,7a-hexahydro-2H-pyrrolo[2,3-c]pyridin-1-yl]pyridazin-3-yl]-3-hydroxy-5-methyl-benzonitrile;
3-Hydroxy-4-[4-methyl-6-(6-methyl-3,3a,4,5,7,7a-hexahydro-2H-pyrrolo[2,3-c]pyridin-1-yl)pyridazin-3-yl]benzonitrile;
4-[6-[(3aS,7aR)-6-Methyl-3,3a,4,5,7,7a-hexahydro-2H-pyrrolo[2,3-c]pyridin-1-yl]-4-methyl-pyridazin-3-yl]-3-hydroxy-benzonitrile or 4-[6-[(3aR,7aS)-6-methyl-3,3a,4,5,7,7a-hexahydro-2H-pyrrolo[2,3-c]pyridin-1-yl]-4-methyl-pyridazin-3-yl]-3-hydroxy-benzonitrile;
4-[6-[(3aR,7aS)-6-Methyl-3,3a,4,5,7,7a-hexahydro-2H-pyrrolo[2,3-c]pyridin-1-yl]-4-methyl-pyridazin-3-yl]-3-hydroxy-benzonitrile or 4-[6-[(3aS,7aR)-6-methyl-3,3a,4,5,7,7a-hexahydro-2H-pyrrolo[2,3-c]pyridin-1-yl]-4-methyl-pyridazin-3-yl]-3-hydroxy-benzonitrile;
4-[4-Ethyl-6-[[(3R)-1-ethyl-3-piperidyl]amino]pyridazin-3-yl]-3-hydroxy-benzonitrile;
and pharmaceutically acceptable salts thereof.

Most preferred examples of compounds of formula Ib as described herein are selected from
4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile;
and pharmaceutically acceptable salts thereof.

Also most preferred examples of compounds of formula Ib as described herein are selected from
4-[6-[(3aS,7aR)-6-Ethyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aR,7aS)-6-ethyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aR,7aS)-6-Ethyl-3,3a,4,5,7,7a-hexahydro-2H-pyrrolo[2,3-c]pyridin-1-yl]pyridazin-3-yl]-3-hydroxy-5-methyl-benzonitrile or 4-[6-[(3aS,7aR)-6-ethyl-3,3a,4,5,7,7a-hexahydro-2H-pyrrolo[2,3-c]pyridin-1-yl]pyridazin-3-yl]-3-hydroxy-5-methyl-benzonitrile;
3-Hydroxy-4-[4-methyl-6-(6-methyl-3,3a,4,5,7,7a-hexahydro-2H-pyrrolo[2,3-c]pyridin-1-yl)pyridazin-3-yl]benzonitrile;
4-[6-[(3aS,7aR)-6-Methyl-3,3a,4,5,7,7a-hexahydro-2H-pyrrolo[2,3-c]pyridin-1-yl]-4-methyl-pyridazin-3-yl]-3-hydroxy-benzonitrile or 4-[6-[(3aR,7aS)-6-methyl-3,3a,4,5,7,7a-hexahydro-2H-pyrrolo[2,3-c]pyridin-1-yl]-4-methyl-pyridazin-3-yl]-3-hydroxy-benzonitrile;
4-[6-[(3aR,7aS)-6-Methyl-3,3a,4,5,7,7a-hexahydro-2H-pyrrolo[2,3-c]pyridin-1-yl]-4-methyl-pyridazin-3-yl]-3-hydroxy-benzonitrile or 4-[6-[(3aS,7aR)-6-methyl-3,3a,4,5,7,7a-hexahydro-2H-pyrrolo[2,3-c]pyridin-1-yl]-4-methyl-pyridazin-3-yl]-3-hydroxy-benzonitrile;
and pharmaceutically acceptable salts thereof.

An embodiment of the present invention provides compounds of formula I, wherein the compound of formula I is a compound of formula Ib

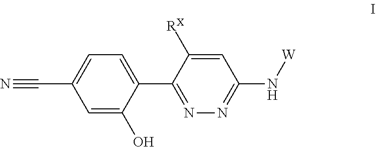

wherein
R^{X} is alkyl;
W is a substituted 4-6-membered-cycloalkyl ring or a substituted heterocycle of ring system A, wherein substituted cycloalkyl is substituted with 1 or 2 substituents selected from OH, halo, and alkyl, and ring system A is

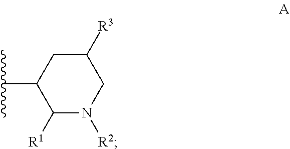

wherein
   R¹ is H;
   R² is alkyl; or
   R¹ and R², together with the atoms to which they are bonded, form a 5-membered ring; and
   R³ is OH or halo;
and pharmaceutically acceptable salts thereof.

An embodiment of the present invention provides compounds according to formula I as described herein, wherein R^{X} is methyl of ethyl.

An embodiment of the present invention provides compounds according to formula I as described herein, wherein R^{X} is methyl.

An embodiment of the present invention provides compounds according to formula I as described here, wherein W is selected from methylcyclobutanol, cyclohexanol, or ring system A, wherein
R¹ is H;
R² is alkyl; or
R¹ and R² bond together to form a 5-membered ring; and
R³ is H, OH or halo.

An embodiment of the present invention provides compounds according to formula I as described here, wherein W is ring system A, wherein
R¹ is H;
R² is methyl or ethyl; or
R¹ and R², together with the atoms to which they are bonded, form a 5-membered ring; and
R³ is H.

An embodiment of the present invention provides compounds according to formula I as described here, wherein W is ring system A, wherein
R¹ is H;
R² is ethyl; and
R³ is H.

An embodiment of the present invention provides compounds according to formula I as described here, wherein W is selected from
i. Methylcyclobutanol
ii. Cyclohexanol
iii. 1-Ethylpiperdine
iv. 1-Methylpiperdine
V. 1-methyl-3-fluoro-piperidine
vi. 1-methylpiperidin-3-ol
vii. 1,2,3,5,6,7,8,8a-octahydroindolizine.

An embodiment of the present invention provides compounds according to formula I as described here, wherein the compound is selected from
4-[6-[[(3R)-1-Ethyl-3-piperidyl]amino]-4-methyl-pyridazin-3-yl]-3-hydroxy-benzonitrile; formic acid; and
4-[6-[[(3R)-1-Ethyl-3-piperidyl]amino]-4-methyl-pyridazin-3-yl]-3-hydroxy-benzonitrile;
and pharmaceutically acceptable salts thereof.

Particular example of compounds of formula I as described herein is 4-[6-[[(3R)-1-Ethyl-3-piperidyl]amino]-4-methyl-pyridazin-3-yl]-3-hydroxy-benzonitrile, and pharmaceutically acceptable salts thereof.

Particular example of compounds of formula I as described herein is 4-[6-[[(3R)-1-Ethyl-3-piperidyl]amino]-4-methyl-pyridazin-3-yl]-3-hydroxy-benzonitrile; formic acid, and pharmaceutically acceptable salts thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula Ib may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula Ib is formulated in an acetate buffer, at pH 5. In another embodiment, the compound of formula Ib is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004: Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula Ib and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula Ib as described herein for use as a therapeutically active substance.

An embodiment of the present invention is a compound according to formula Ib as described herein for use in the treatment or prevention of a disease, disorder or condition, wherein the disease, disorder or condition is responsive to NLRP3 inhibition.

An embodiment of the present invention is a compound according to formula Ib as described herein for the treatment or prophylaxis of a disease, disorder or condition, wherein the disorder or condition is responsive to NLRP3 inhibition.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to formula Ib as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

An embodiment of the present invention is the use of a compound according to formula Ib as described herein in the treatment or prophylaxis of a disease, disorder or condition, wherein the disease, disorder or condition is responsive to NLRP3 inhibition.

An embodiment of the present invention is the use of a compound according to formula Ib as described herein in the treatment or prophylaxis of a disease, disorder or condition selected from Alzheimer's disease and Parkinson's disease.

An embodiment of the present invention is the use a compound according to formula Ib as described herein for use in the treatment or prophylaxis of a disease, disorder or condition selected from Asthma or COPD.

An embodiment of the present invention is a compound according to formula Ib as described herein for the treatment or prophylaxis of a disease, disorder or condition selected from Alzheimer's disease and Parkinson's disease.

An embodiment of the present invention is a compound according to formula Ib as described herein for the treatment or prophylaxis of a disease, disorder or condition selected from Asthma or COPD.

An embodiment of the present invention is the use of a compound according to formula Ib as described herein for preparation of a medicament for the treatment or prophylaxis of a disease, disorder or condition selected from Alzheimer's disease and Parkinson's disease.

An embodiment of the present invention is the use of a compound according to formula Ib as described herein for the preparation of a medicament for the treatment or prophylaxis of a disease, disorder or condition selected from Asthma or COPD.

An embodiment of the present invention is a method of treatment or prophylaxis of a disease, disorder or condition selected from Alzheimer's disease and Parkinson's disease, which method comprises administering an effective amount of a compound according to formula Ib as described herein.

An embodiment of the present invention is a method of treatment or prophylaxis of a disease, disorder or condition selected from Asthma or COPD, which method comprises administering an effective amount of a compound according to formula Ib as described herein.

An embodiment of the present invention relates to a method of inhibiting NLRP3, which method comprises administering an effective amount of a compound according to formula Ib as described herein.

Also an embodiment of the present invention are compounds of formula Ib as described herein, when manufactured according to any one of the described processes.

An embodiment of the present invention is a pharmaceutical composition comprising a compound according to formula Ib as described herein and a therapeutically inert carrier.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use of a compound according to formula I as described herein in the treatment or prophylaxis of a disease, disorder or condition selected from Alzheimer's disease and Parkinson's disease.

An embodiment of the present invention is the use a compound according to formula I as described herein for use in the treatment or prophylaxis of a disease, disorder or condition selected from Asthma or COPD.

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Asthma or COPD.

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for the preparation of a medicament for the treatment or prophylaxis of a disease, disorder or condition selected from Asthma or COPD.

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Asthma or COPD, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27: Zhen Xie & Gang Zhao, Neuroimmunology Neuroinflammation, 2014, 1(2), 60-65; Mattia Cocco et al., Journal of Medicinal Chemistry, 2014, 57(24), 10366-10382: T. Satoh et al., Cell Death & Disease, 2013, 4, e644). Therefore, it is anticipated that inhibitors of NLRP3 will block pyroptosis, as well as the release of pro-inflammatory cytokines (e.g. IL-1B) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜106 cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 10 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300 xg for 3 mins and remove supernatant
7. Then add 50 μl of resazurin (Sigma #R⁷⁰¹⁷) (FAC 100 UM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300 xg for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

hERG Screening Assay

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂) 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

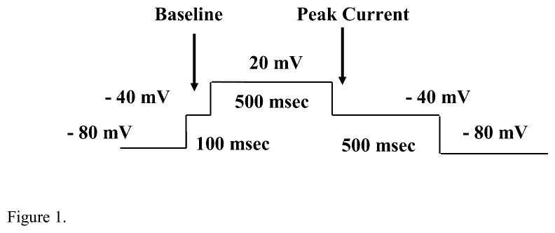

Cells were held at a resting voltage of −80 mV and they were stimulated by a voltage pattern shown in FIG. 1 (pulse pattern used to elicit outward K+ current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

$I ( C ) = \frac{100}{1 + {( C / IC 50 )}^{h}}$

| | |
|---|---|
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter Logistic Model (fit=(A+(B/(1+((x/C)\hat{ }D)))), where A=0 and B=100).

### Brain Penetration;

Brain penetration was studied in rat by measuring drug concentrations in plasma, brain, and cerebrospinal fluid (CSF) samples after peroral drug administration. Unbound brain concentrations were estimated through kinetic lipid membrane binding assays and ex vivo partitioning experiments. The unbound partitioning coefficients from brain or CSF to plasma (kp,u,u) were determined by correlating unbound brain or CSF concentration to the plasma exposure corrected for plasma protein binding.

### Transcellurlar P-gp Assay;

The general assay uses transfected LLC-PKI cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures: pooled (n=5 for male and n=5 for female for human)] with stromal mouse fibroblasts (negative control: pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/ml hydrocortisone and 0.004 mg/mL insulin, to a final suspension density of 1×106 cells/mL. The incubation was performed fully automatically with Liquid Handling System (Tecan) equipped with a CO₂ incubator with an orbital shaker. After the addition of a test compound at e.g. 1 μM to the wells (1×105 cells/well), the 96-well hepatocyte suspension culture plates are incubated in a 5% CO₂ at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

TABLE 1
NLRP3 inhibitory activity

| | THP-1 pyroptosis | Human whole blood IL-1β |
|---|---|---|
| | Assay | Assay IC₅₀ |
| Example No. | IC₅₀ (nM) | (nM) |
| 1 | 3.1 | 3.2 |
| 2 | 1.1 | 5.0 |
| 3 | 73.1 | 51.8 |
| 4 | 822.6 | |
| 5A | 1.2 | 4.6 |
| 5B | 47.0 | 326.2 |
| 6A | 3.9 | 14.9 |
| 6B | 763.2 | |
| 7 | 1.8 | 6.6 |
| 7B | 1.8 | 5.6 |
| 7A | 276.7 | |
| 8 | 5.6 | 9.9 |

In the drug development process of small molecules, one of the most frequent adverse side effects, leading to the failure of drugs, is the cardiac arrhythmias. Such failure is often related to the capacity of the drug to inhibit the human ether-à-go-go-related gene (hERG) cardiac potassium channel. Having no or low inhibition of the hERG cardiac potassium channel is therefore considered as beneficial.

TABLE 2
Inhibitory activity at hERG

| Example | | | hERG assay | |
|---|---|---|---|---|
| No. | Structure | Name | IC₅₀ (μM) | |
| 1 | 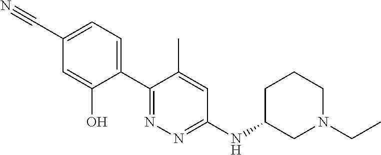 [SMILES: CCN1CCC[C@@H](Nc2cc(C)c(-c3ccc(C#N)cc3O)nn2)C1] 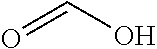 | 4-[6-[[(3R)-1-Ethyl-3- piperidyl]amino]-4- methyl-pyridazin-3-yl]- 3-hydroxy-benzonitrile; formic acid | >20 | μM |
| RE-A* | 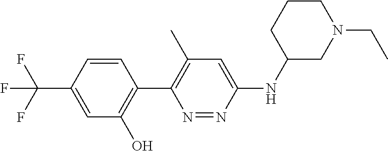 | 2-[6-[(1-ethyl-3- piperidyl)amino]- 4-methyl-pyridazin-3- yl]-5- (trifluoromethyl)phenol | 1.2 | μM |
| RE-B* | 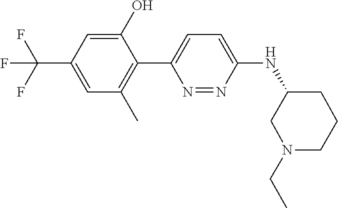 | 3-methyl-2-[6-[[(3R)-1- ethyl-3- piperidyl]amino]pyridazin- 3-yl]-5- (trifluoromethyl)phenol | 9.5 | μM |

*RE-A and *RE-B were synthesized as or in analogy of WO20200234715.

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### Experimental Methods

### Abbreviations:

| | |
|---|---|
| DEA | N,N-Diethylamine |
| DIPEA | N,N-Diisopropylethylamine |
| h | hour(s) |
| HPLC | high performance liquid chromatography |
| MeCN | acetonitrile |
| MeOH | methanol |
| NMP | N-Methyl-2-pyrrolidone |
| SFC | Supercritical fluid chromatography |
| tBME | 2-Methoxy-2-methylpropane |
| r.t. | room temperature |

### EXAMPLES

All examples and intermediates were prepared under nitrogen atmosphere if not specified otherwise.

### Preparative HPLC Conditions:

The sample was dissolved in 10 mL with DMSO, filtered and purified by reversed phase preparative HPLC (Gilson) using a Phenomenex Gemini NC-C18 prep column, 110 Å, 5 μm, 30 mm×150 mm, flow rate 40 mL min-1 eluting with a 0.1% formic acid in water-MeCN gradient over 6 min. At-column dilution pump gives 5 mL min-1 MeCN for 1.2 min. Gradient information: 0.0-1 min, 5% MeCN: 1-7.5 min, ramped from 5% MeCN to 15.8% MeCN: 7.5-7.6 min, ramped from 15.8% MeCN to 100% MeCN: 7.6-10.9 min, held at 100% MeCN. The clean fractions were evaporated in a freeze dried.

### Example 1

### 4-[6-[[(3R)-1-Ethyl-3-piperidyl]amino]-4-methyl-pyridazin-3-yl]-3-hydroxy-benzonitrile; formic acid

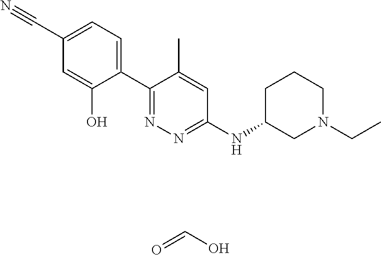 [SMILES: CCN1CCC[C@@H](Nc2cc(C)c(-c3ccc(C#N)cc3O)nn2)C1 ; O=CO]

### Intermediate A1: 6-Chloro-N-[(3R)-1-ethyl-3-piperidyl]-5-methyl-pyridazin-3-amine

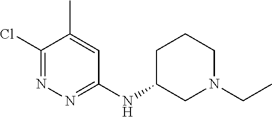 [SMILES: CCN1CCC[C@@H](Nc2cc(C)c(Cl)nn2)C1]

3,6-dichloro-4-methylpyridazine (CAS #19064-64-3, 3.0 g, 18.4 mmol, 1.0 eq) and DIPEA (8.02 mL, 46.01 mmol, 2.5 eq) and (3R)-1-ethylpiperidin-3-amine (CAS #1020396-26-2, 2.95 g, 23.01 mmol, 1.25 eq) were dissolved in NMP (30 mL) and the reaction mixture stirred at 120° C. for 6 days. The reaction mixture was diluted with EtOAc (200 mL) and washed with brine (2×150 mL) and 10 wt % aqueous LiCl (2×150 mL), then dried using a phase separator and concentrated in vacuo. The resulting residue was purified by chromatography on silica gel (24 g column, 0-10% (0.7 N NH3 in MeOH)/CH₂C₁₂) to afford the title compound (1.21 g, 4.75 mmol, 17% yield) as an orange solid (˜3:1 mixture of the desired product and its regioisomer 6-chloro-N-[(3R)-1-ethyl-3-piperidyl]-4-methyl-pyridazin-3-amine). LCMS m/z 255.3 (M+H)⁺(ES+).

### Intermediate B1: 4-[6-[[(3R)-1-Ethyl-3-piperidyl]amino]-4-methyl-pyridazin-3-yl]-3-methoxy-benzonitrile

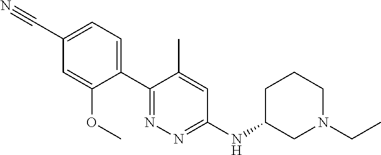 [SMILES: CCN1CCC[C@@H](Nc2cc(C)c(-c3ccc(C#N)cc3OC)nn2)C1]

6-Chloro-N-[(3R)-1-ethyl-3-piperidyl]-5-methyl-pyridazin-3-amine (Intermediate A1, 200.0 mg, 0.390 mmol, 1.0 eq), 4-cyano-2-methoxyphenylboronic acid (CAS #1256345-67-1, 196 mg, 1.11 mmol, 2.2 eq) and saturated aqueous sodium carbonate (0.5 mL, 0.390 mmol, 1 eq) were suspended in 1,4-dioxane (3 mL) and the reaction mixture was sparged with N2, then evacuated and back-filled with N₂ (3×). XPhos Pd G3 (40.0 mg, 0.050 mmol, 0.09 eq) was added and the reaction mixture placed under N₂, then stirred at 80° C. for 18 h. The reaction mixture was left to cool to room temperature and dry-loaded onto silica gel (5 g). The crude product was purified by column chromatography (SiO₂, 40 g cartridge, 0-10% (0.7 N NH3 in MeOH)/CH₂Cl₂) to give the title compound (184 mg, 0.52 mmol, 93% yield) (˜3:1 mixture with its regioisomer 4-[6-[[(3R)-1-ethyl-3-piperidyl]amino]-5-methyl-pyridazin-3-yl]-3-methoxy-benzonitrile) as a yellow solid. LCMS m/z 352.1 (M+H)+(ES+).

### Example 1: 4-[6-[[(3R)-1-ethyl-3-piperidyl]amino]-4-methyl-pyridazin-3-yl]-3-hydroxy-benzonitrile; formic acid

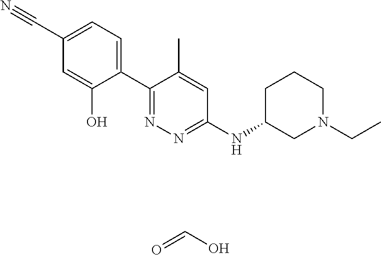 [SMILES: CCN1CCC[C@@H](Nc2cc(C)c(-c3ccc(C#N)cc3O)nn2)C1 ; O=CO]

A solution of 4-[6-[[(3R)-1-ethyl-3-piperidyl]amino]-4-methyl-pyridazin-3-yl]-3-methoxy-benzonitrile (Intermediate B1) (172.0 mg, 0.49 mmol, 1.0 eq) (Note: Starting material is a ˜3:1 mixture of methyl pyridazine regioisomers) in CH₂Cl₂ (8 mL) was treated with boron tribromide 1M in CH₂C₁₂ (1.7 mL, 1.70 mmol, 3.47 eq) dropwise at 0° C. After 30 min the mixture was allowed to warm and stir at room temperature for 2 h. The reaction mixture was cooled to 0° C. and boron tribromide IM in CH₂C12 (1.35 mL, 1.35 mmol, 3.94 eq) was added and reaction mixture was then allowed to warm to r.t. and stir for 1.5 h. The reaction mixture was quenched with 0.7M NH3 MeOH (˜15 mL) and left to stir for 30 min and concentrated under reduced pressure to provide the crude product. This was submitted for preparative HPLC separation, to afford the title compound (50 mg, 0.13 mmol, 25% yield) as a light brown solid. LCMS: m/z 338.3 (M+H)+(ES+): 336.4 (M−H)⁻ (ES−).

### Example 2

### 4-[6-[[(3R)-1-Ethyl-3-piperidyl]amino]pyridazin-3-yl]-3-hydroxy-5-methyl-benzonitrile

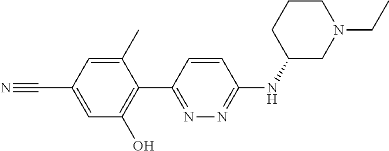 [SMILES: CCN1CCC[C@@H](Nc2ccc(-c3c(C)cc(C#N)cc3O)nn2)C1]

### Intermediate 2A: 6-Chloro-N-[(3R)-1-ethyl-3-piperidyl]pyridazin-3-amine

In a sealed tube, to a yellow solution of commercially available 3,6-dichloropyridazine (CAS #141-30-0, 500 mg, 3.36 mmol, 1.0 eq) and commercially available [(3R)-1-ethyl-3-piperidyl]amine (CAS #1020396-26-2, 516.7 mg, 4.03 mmol, 1.2 eq) in N-methyl-2-pyrrolidinone (2.83 mL) was added under stirring N,N-Diisopropylethylamine (1.5 mL, 8.59 mmol, 2.56 eq) at room temperature. The yellow reaction mixture was stirred at 120° C. overnight (16 hours). The reaction mixture was cooled to room temperature and extracted with ˜70 mL ethyl acetate and ˜10 mL and aq. 5% LiCl-solution. The aqueous layer was back extracted with ˜70 mL ethyl acetate. The organic layers were washed twice with ˜10 mL aq. 5% LiCl-solution, once with ˜10 mL water and once with ˜10 mL brine. The combined organic layers were dried over sodium sulfate, filtered and concentrated in vacuo. The crude product was adsorbed on ISOLUTE HM-N and purified by flash chromatography (silica gel, gradient 0% to 100% (dichloromethane:methanol:NH₄OH 9:1:0.05) in dichloromethane) to afford the title compound (542 mg, 66%) as off-white solid. LCMS: m/z 241.1 [M+H]⁺, ESI pos.

### Intermediate 2B: 4-Amino-3-methoxy-5-methyl-benzonitrile

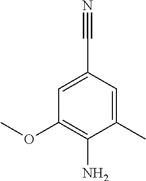

Two Batches were Carried Out in Parallel.

To a solution of commercially available 4-bromo-2-methoxy-6-methylbenzenamine (CAS #348169-39-1, 25.0 g, 115 mmol, 1.00 eq) in DMF (250 mL) was added Zn(CN)₂ (13.5 g, 115 mmol, 7.34 mL, 1.00 eq) and Pd(PPh₃)₄ (66.8 g, 57.8 mmol, 0.50 eq). The reaction mixture was stirred at 100° C. for 12 hrs. The reaction mixture was poured into water (1.50 L) and extracted with ethyl acetate (1 L×3). The organic phase was washed with brine (1 L×3), dried over Na₂SO₄, filtered and concentrated under reduced pressure to give a residue, which was purified by column chromatography (SiO₂, petroleum ether/ethyl acetate=100/1 to 0/1) to give the title compound (28.0 g, 75% yield) as a yellow solid. 1H NMR (DMSO-d₆) δ 7.05 (s, 2H), 5.47 (bs, 2H), 3.81 (s, 3H), 2.09 (s, 3H).

### Intermediate 2C: 4-Bromo-3-methoxy-5-methyl-benzonitrile

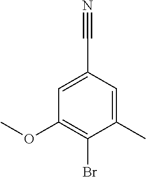

To a solution of CuBr (46.4 g, 323 mmol, 9.86 mL, 1.50 eq) in MeCN (180 mL) was added t-BuONO (33.3 g, 323 mmol, 38.5 mL, 1.50 eq) and stirred at 65° C. Then solution of aforementioned Intermediate 2B 4-bromo-3-methoxy-5-methyl-benzonitrile (35.0 g, 215 mmol, 1.00 eq) in MeCN (180 mL) was added at 65° C. The mixture was stirred at 65° C. for 3.5 hrs. After completion, sat. aq. Na₂SO₃ (400 mL) and sat. aq. NH4Cl (200 mL) was added to the mixture and extracted with ethyl acetate (500 mL×3). The organic phase was washed with brine (500 mL×2), dried over Na₂SO₄, filtered and concentrated under reduced pressure to give a residue. The residue was purified by column chromatography (SiO₂, petroleum ether/ethyl acetate=100/1 to 0/1, Rf=0.75) to give the title compound (20.7 g, 42% yield) as a white solid. 1H NMR (DMSO-d₆) δ 7.43, 7.40 (2s, 1H each), 3.90 (s, 3H), 2.37 (s, 3H).

### Intermediate 2D: 3-Methoxy-5-methyl-4-(4,4,5,5-tetramethyl-1,3,2-dioxaborolan-2-yl)benzonitrile

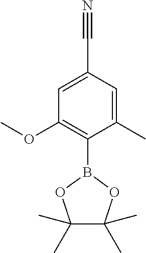

To a solution of aformentioned 4-bromo-3-methoxy-5-methyl-benzonitrile (18.0 g, 79.6 mmol, 1.00 eq) in DMF (180 mL) was added B₂Pin₂ (30.3 g, 119 mmol, 1.50 eq) and AcOK (35.1 g, 358 mmol, 4.50 eq). The mixture was stirred at 20° C. for 0.5 hr and Pd(dppf)C₁₂·CH₂Cl₂ (13.0g, 15.9 mmol, 0.20 eq) was added. The mixture was stirred at 100° C. for 12 hrs. The mixture was filtered with diatomite and diluted with H₂O (500 mL) and extracted with ethyl acetate (800 mL×3). The organic phase was washed with brine (800 mL×3), dried over Na₂SO₄, filtered and concentrated under reduced pressure to give a residue. The residue was purified by column chromatography (SiO₂, petroleum ether/ethyl acetate=100/1 to 1/1, Rf=0.30) to give the title compound (18.0 g, 83% yield) as a white solid. 1H NMR (DMSO-d₆) δ 7.22, 7.21 (2s, 1H each), 3.75 (s, 3H), 2.27 (s, 3H), 1.30 (s, 12H).

### Intermediate 2E: (4-Cyano-2-hydroxy-6-methyl-phenyl)boronic acid

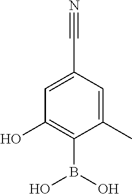

A solution of aforementioned 3-methoxy-5-methyl-4-(4,4,5,5-tetramethyl-1,3,2-dioxaborolan-2-yl)benzonitrile (17.0 g, 96.0 mmol, 1.00 eq) in dichloromethane (170 mL) was cooled to 0° C. and BBr₃ (38.9 g, 155 mmol, 2.50 eq) was added dropwise at 0° C. The mixture was stirred at 0° C. for 0.5 hr. The mixture was poured into H₂O (200 mL), filtered, and the cake was collected and triturated with EtOAc (20 mL) to give the title compound (4.67 g, 42% yield) as a gray solid. LCMS: m/z 178.1 [M+H]⁺, ESI pos.

### Example 2: 4-[6-[[(3R)-1-Ethyl-3-piperidyl]amino]pyridazin-3-yl]-3-hydroxy-5-methyl-benzonitrile

In sealed tube, to a mixture of aforementioned intermediate A2 6-chloro-N-[(3R)-1-ethyl-3-piperidyl]pyridazin-3-amine (80 mg, 0.332 mmol, 1.00 eq) and intermediate 2E (4-cyano-2-hydroxy-6-methyl-phenyl)boronic acid (128 mg, 0.665 mmol, 2.00 eq) in 1,4-dioxane, extra dry (2 mL) and water (1 mL) was added under stirring at room temperature (23° C.) potassium carbonate (206.7 mg, 1.50 mmol, 4.50 eq). The orange reaction mixture was bubbled argon through for 3 minutes and afterwards 1, l′-bis(diphenylphosphino)ferrocene-palladium(II)dichloride dichloromethane complex (40.7 mg, 0.05 mmol, 0.150 eq) was added under stirring at room temperature (23° C.). The orange reaction mixture was sealed and stirred at 95° C. (oil bath) for 16 hours (overnight). After reaction completion, mixture was cooled to room temperature and extracted with dichloromethane twice (2×˜ 20 mL) and saturated NH₄Cl-solution (˜20 mL). The organic layers were washed with water (˜10 mL) and brine (˜10 mL). The aqueous phases were back extracted with dichloromethane (˜20 mL). The combined organic extracts were dried over sodium sulfate, filtered off and concentrated in vacuo. The crude product was absorbed on ISOLUTE HN-M and purified by flash chromatography (SiO₂: gradient 0%-20% dichloromethane:methanol:NH₄OH (v/v) 110:10:1 in dichloromethane) followed by further purification by preparative HPLC to afford the title compound (51.7 mg, 46%) as white powder. LCMS: m/z 338.2 [M+H]⁺, ESI pos.

### Example 3

### 4-[6-[[(3R)-1-Ethyl-3-piperidyl]amino]-4-(trifluoromethyl)pyridazin-3-yl]-3-hydroxy-benzonitrile

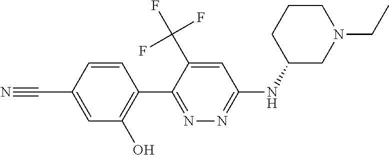 [SMILES: CCN1CCC[C@@H](Nc2cc(C(F)(F)F)c(-c3ccc(C#N)cc3O)nn2)C1]

### Intermediate 3 Å: tert-Butyl (3R)-3-[[6-chloro-5-(trifluoromethyl)pyridazin-3-yl]amino]pipe-ridine-1-carboxylate and tert-butyl (3R)-3-[[6-chloro-4-(trifluoromethyl)pyridazin-3-yl]amino]piperidine-1-carboxylate

A neat mixture of commercially available 3,6-dichloro-4-(trifluoromethyl)pyridazine (CAS #1057672-68-0, 1.72 g, 7.93 mmol, 1.0 eq) and commercially available (3R)-3-aminopiperidine-1-carboxylic acid tert-butyl ester (CAS #188111-79-7, 3.18 g, 15.9 mmol, 2.0 eq) in N,N-diisopropylethylamine (3.46 mL, 19.8 mmol, 2.5 eq) was stirred at 130° C. in a sealed tube for 24 hours. The warm mixture (˜50° C.) was poured into an Erlenmeyer with ethyl acetate (100 mL) which was used to transfer the oily mixture, and water (100 mL) was added. It was stirred at room temperature for 20 min to dissolve all ingredients. Extraction was undertaken with ethyl acetate/water and finally brine. The residue was purified by flash chromatography (SiO₂: 0-50% ethyl acetate in heptane) to give the title compound (first regioisomer) (1.67 g, 55%) as light yellow foam as well as the second regiosisomer (1.07 g, 35% yield) as light yellow oil. LCMS: m/z 381.1 ([{35Cl}M+H]⁺), 383.1 ([{37C₁}M+H]⁺), ESI pos.

### Intermediate 3B: 6-Chloro-N-[(3R)-3-piperidyl]-5-(trifluoromethyl)pyridazin-3-amine;hydro-chloride

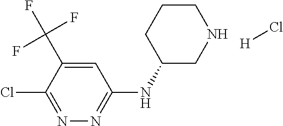

To a solution of aforementioned tert-butyl (3R)-3-[[6-chloro-5-(trifluoromethyl)pyridazin-3-yl]amino]pipe-ridine-1-carboxylate intermediate 3A (first regioisomer) (1.67 g, 4.39 mmol, 1.00 eq) in dichloromethane (20 mL) and methanol (10 mL) was added via syringe 4 M HCl (in dioxane) (13.2 g, 10.9 mL, 43.9 mmol, 10.0 eq). The clear, yellow reaction solution was stirred at r.t. for 16 hours. After complete conversion, the reaction mixture was concentrated in vacuo to afford the title compound (1.51 g, 98% yield) as light yellow foam. The compound was used without further purification in the next step. LCMS: m/z 281.1 ([{35Cl}M+H]+), 283.1 ([{37Cl}M+H]+), ESI pos.

### Intermediate 3C: 6-Chloro-N-[(3R)-1-ethyl-3-piperidyl]-5-(trifluoromethyl)pyridazin-3-amine

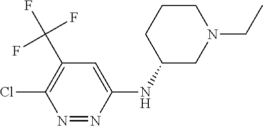 [SMILES: CCN1CCC[C@@H](Nc2cc(C(F)(F)F)c(Cl)nn2)C1]

To a suspension of aforementioned 6-chloro-N-[(3R)-3-piperidyl]-5-(trifluoromethyl)pyridazin-3-amine;hydro-chloride intermediate 3B (1.51 g, 4.29 mmol, 1.00 eq) in dry dichloromethane (30 mL) was added acetaldehyde (472 mg, 597 μL, 10.7 mmol, 2.5 eq) followed by sodium acetate (879 mg, 10.7 mmol, 2.5 eq) under ice-bath cooling. Then sodium triacetoxyborohydride (1.63 g, 7.71 mmol, 1.8 eq) was added at 0° C. The reaction mixture was stirred at 0° C. for 15 min and at r.t. for 2 hours (light yellow suspension). After complete conversion, the reaction mixture was carefully basified with aq. NaHCO₃-solution (50 mL) and then extracted with dichloromethane (3×80 mL). The combined organic layers were dried over sodium sulfate, filtered and concentrated in vacuo. The crude product (1.4 g) was adsorbed on ISOLUTE HM-N and purified by flash chromatography [silica gel, gradient 0% to 100% (dichloromethane:methanol:NH₄OH 110:10:1) in dichloromethane] to afford the title compound (1.03 g, 76% yield) as light brown oil. LCMS: m z 309.1 ([{35Cl}M+H]⁺), 311.0 ([{37C1}M+H]⁺), ESI pos.

### Example 3: 4-[6-[[(3R)-1-Ethyl-3-piperidyl]amino]-4-(trifluoromethyl)pyridazin-3-yl]-3-hydroxy-benzonitrile

In a sealed tube aforementioned 6-chloro-N-[(3R)-1-ethyl-3-piperidyl]-5-(trifluoromethyl)pyridazin-3-amine intermediate 3C (143 mg, 454 μmol, 1.0 eq) and commercially available (4-cyano-2-hydroxy-phenyl)boronic acid (no CAS #, 130.9 mg, 771.7 μmol, 1.70 eq) were dissolved in 1,4-dioxane (5 mL) and water (2.5 mL). Under stirring potassium carbonate (282.3 mg, 2.04 mmol, 4.5 eq) was added at r.t. followed by the addition of 1, l′-bis(diphenylphosphino)ferrocenepalladium(II)dichloride dichloromethane complex (55.6 mg, 68.09 μmol, 0.15 eq) under an atmosphere of argon. Then the mixture was stirred at 95° C. overnight. The dark-brown mixture was cooled to r.t. and extracted with water, ethyl acetate, brine and ammonium chloride. The aqueous layers were back extracted with ethyl acetate twice. The combined organic layers were washed with brine, then dried over sodium sulfate, filtered and concentrated in vacuo. The crude was purified by RP-HPLC (C₁₈, column: YMC-triart, 12 nm, 5 μm, 100×30 mm, ELSD, acetonitrile/water+0.1% triethylamine) to give the title compound (24 mg, 13% yield) as a white powder. LCMS: m/z 392.2 [M+H]⁺, ESI pos.

### Example 4

### 3-Hydroxy-4-[6-[(3-hydroxy-3-methyl-cyclobutyl)amino]-4-methyl-pyridazin-3-yl]benzonitrile

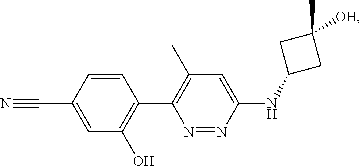 [SMILES: Cc1cc(N[C@H]2C[C@@](C)(O)C2)nnc1-c1ccc(C#N)cc1O]

A mixture of 3-[(6-chloro-5-methyl-pyridazin-3-yl)amino]-1-methyl-cyclobutanol (CAS #2557359-89-2, 70.0 mg, 0.28 mmol, 1.00 eq, 90% purity), commercially available (no CAS #, 4-cyano-2-hydroxy-phenyl)boronic acid (95 mg, 0.58 mmol, 2.11 eq), potassium carbonate (195 mg, 1.41 mmol, 5.10 eq) and 1,1′-bis(diphenylphosphino)ferrocene-palladium(II) dichloride dichloromethane complex (45 mg, 0.06 mmol, 0.20 eq) in 1,4-dioxane (2.2 mL) and water (1.1 mL) was flushed with argon and stirred at 95° C. for 16 hours. The reaction mixture was cooled to r.t. and extracted with ethyl acetate and water. The aqueous layer was back extracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 10% methanol in dichloromethane). All fractions containing product were combined and concentrated in vacuo. The residue was adsorbed on ISOLUTE HM-N and purified a second time by flash chromatography (SI-amine, gradient 0% to 10% methanol in ethyl acetate). All fractions containing product were combined and concentrated in vacuo to afford the title compound (25 mg, 28% yield) as an off-white solid. LCMS: m/z 311.2 [M+H]⁺, ESI pos.

### Examples 5A and 5B

### 4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile and 4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile

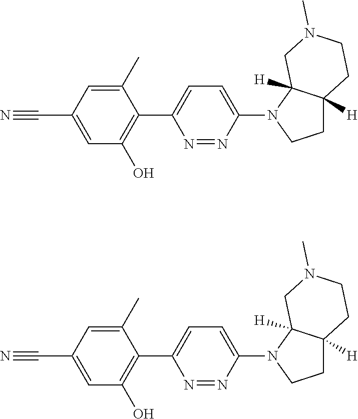 [SMILES: Cc1cc(C#N)cc(O)c1-c1ccc(N2CC[C@H]3CCN(C)C[C@H]32)nn1 ; Cc1cc(C#N)cc(O)c1-c1ccc(N2CC[C@@H]3CCN(C)C[C@@H]32)nn1]

### Intermediate 5 Å: tert-Butyl 1-(6-chloropyridazin-3-yl)-3,3a,4,5,7,7a-hexahydro-2H-pyrrolo[2,3-c]pyridine-6-carboxylate

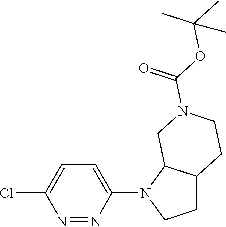 [SMILES: CC(C)(C)OC(=O)N1CCC2CCN(c3ccc(Cl)nn3)C2C1]

To a mixture of commercially available 3,6-dichloropyridazine (CAS #141-30-0, 413 mg, 2.69 mmol, 1.00 eq) and commercially available 6-boc-octahydropyrrolo[2,3-c]pyridine (CAS #1286755-20-1, 752.9 mg, 3.23 mmol, 1.20 eq) in N-methyl-2-pyrrolidinone (3 mL) was added N-ethyldiisopropylamine (1.14 mL, 6.72 mmol, 2.50 eq). The reaction mixture was stirred at 120° C. overnight. After complete conversion, the brown reaction mixture was cooled to room temperature, poured into ice water and brine, and extracted with ethyl acetate:tBME (v/v) 1:1 (3×80 mL). The organic layers were washed with water (80 mL) and brine (80 ml). The aqueous layers were reextracted with ethyl acetate:tBME (v/v) 1:1 (80 mL). The combined organic extracts were dried over sodium sulfate, filtered off and concentrated in vacuo. The residue was purified by flash chromatography (SiO₂: 0-90% ethyl acetate in heptane) to give the title compound (865 mg, 95%) as light yellow oil. LCMS: m/z 339.2 ([{35Cl}M+H]+), 341.1 ([{37C1}M+H]+), ESI pos.

### Intermediate 5B: 1-(6-Chloropyridazin-3-yl)-2.3.3a, 4,5,6,7,7a-octahydropyrrolo[2,3-c]pyridine;hydrochloride

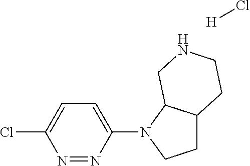

To a solution of aforementioned intermediate 5A (865 mg, 2.55 mmol, 1.00 eq) in dichloromethane, extra dry (20 mL) was added at room temperature 4 M HCl (6.38 mL, 25.5 mmol, 10.0 eq) dropwise. The reaction mixture was stirred at 23° C. for 5 hours. After complete conversion, the mixture was concentrated in vacuo to give the crude title compound as hydrogen chloride (733 mg) as light yellow foam which was used without further purification in the next step. LCMS: m/z 239.2 ([{35Cl}M+H]+), 241.1 ([{37Cl}M+H]+), ESI pos.

### Intermediate 5C: 1-(6-Chloropyridazin-3-yl)-6-methyl-3,3a,4,5,7,7a-hexahydro-2H-pyrrolo[2,3-c]pyridine

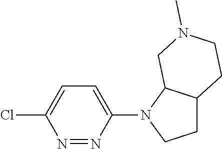 [SMILES: CN1CCC2CCN(c3ccc(Cl)nn3)C2C1]

To a suspension of aforementioned intermediate 5B (733 mg, 2.66 mmol, 1.00 eq) in 1,2-dichloroethane (20 mL) was added Triethylamine (577 μL, 4.13 mmol, 1.552 eq). Let stir at room temperature for 5 min. Formaldehyde, 37% aqueous solution (448.5 mg, 411 μL, 5.53 mmol, 2.08 eq) was added followed by portion-wise addition of sodium triacetoxyborohydride (2.26 g, 10.7 mmol, 4.00 eq). The reaction mixture was stirred at room temperature for 1.5 h. The reaction mixture was carefully quenched with saturated aq. NaHCO₃-solution and extracted four times with dichloromethane. The combined organic layers were dried over sodium sulfate, filtered and concentrated in vacuo to afford the crude title compound (680 mg) as off-white solid which was used without further purification in the next step. LCMS: m/z 253.2 ([{35Cl}M+H]⁺), 255.2 ([{37C1}M+H]⁺), ESI pos.

### Example 5A and 5B: 4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile and 4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile

In a sealed tube, to a yellow solution of aforementioned intermediate 5C (300 mg, 1.19 mmol, 1.00 eq) and aforementioned (4-cyano-2-hydroxy-6-methyl-phenyl)boronic acid intermediate 2E (420.1 mg, 2.37 mmol, 2.00 eq) in 1,4-dioxane (10 mL) and water (2.5 mL) was added under stirring cesium carbonate (1.16 g, 3.56 mmol, 3.00 eq) at room temperature (23° C.). The yellow reaction solution was flushed with argon (balloon) for 3 minutes followed by the addition of XPhos Pd G3 (201 mg, 237 μmol, 0.20 eq) at RT. The yellow reaction mixture was flushed again with argon (balloon) for 2 minutes and stirred at 100° C. (preheated oil bath) for 3 hours. After complete conversion, the orange-yellow reaction mixture was cooled to room temperature, transferred in a separating funnel and extracted with dichloromethane (50 mL) and saturated NH₄Cl-solution (40 mL). The organic phases were washed with water (20 mL) and brine (20 mL). The aqueous phase were back extracted with dichloromethane twice (2×50 mL). The combined organic extracts were dried over sodium sulfate, filtered off and concentrated in vacuo. The crude product was adsorbed on ISOLUTE-HN-M and purified by flash chromatography (SiO₂: 0%-70% dichlormethane:methanol:NH₄OH (v/v) 110:10:1 in CH₂Cl₂) to afford the title product (383 mg, 91%) as orange foam which was directly purified by chiral HPLC (column: chiral IK 5 μm, 250×20 mm; SFC, flow: 80 mL/min, 80 bar, 220 nm, 38% MeOH, 0.2% DEA). The first enantiomer (Example 5 Å) was obtained (rt=4.599 min, 103 mg, 27%) and the second enantiomer (Example 5B) (rt=4.990 min, 157 mg, 41%) both as light brown foam. LCMS: m/z 350.2 [M+H]⁺, ESI pos.

### Reference Example RE-A: 2-[6-[(1-ethyl-3-piperidyl)amino]-4-methyl-pyridazin-3-yl]-5-(trifluoromethyl)phenol

RE-A was synthesized in analogy of WO20200234715.

### Reference Example RE-B: 3-methyl-2-[6-[[(3R)-1-ethyl-3-piperidyl]amino]pyridazin-3-yl]-5-(trifluoromethyl)phenol

RE-B was synthesized as described in WO20200234715.

### Example 6A and 6B

### 4-[6-[(3aS,7aR)-6-Ethyl-3,3a,4,5,7,7a-hexahydro-2H-pyrrolo[2,3-c]pyridin-1-yl]pyridazin-3-yl]-3-hydroxy-5-methyl-benzonitrile and 4-[6-[(3aR,7aS)-6-ethyl-3,3a,4,5,7,7a-hexahydro-2H-pyrrolo[2,3-c]pyridin-1-yl]pyridazin-3-yl]-3-hydroxy-5-methyl-benzonitrile

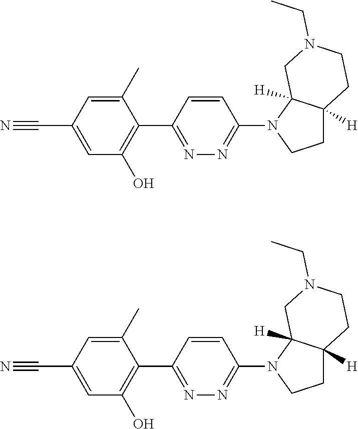 [SMILES: CCN1CC[C@H]2CCN(c3ccc(-c4c(C)cc(C#N)cc4O)nn3)[C@H]2C1 ; CCN1CC[C@@H]2CCN(c3ccc(-c4c(C)cc(C#N)cc4O)nn3)[C@@H]2C1]

### Step A: tert-Butyl 1-(6-chloropyridazin-3-yl)-3,3a,4,5,7,7a-hexahydro-2H-pyrrolo[2,3-c]pyridine-6-carboxylate

To a mixture of 3,6-dichloropyridazine (CAS #141-30-0, 300 mg, 2.01 mmol, 1.00 eq) and 1,2,3,3a,4,5,7,7a-octahydropyrrolo[2,3-c]pyridine-6-carboxylic acid tert-butyl ester (CAS #1196147-27-9, 548 mg, 2.42 mmol, 1.20 eq) in N-methyl-2-pyrrolidinone (2.0 mL) was added N,N-diisopropylethylamine (666 mg, 0.90 mL, 5.15 mmol, 2.56 eq). The reaction mixture was stirred at 120° C. for 16 hours. The reaction mixture was cooled to room temperature and extracted with ethyl acetate and aq. 5% LiCl-solution. The aqueous layer was back extracted with ethyl acetate. The organic layers were washed three times with aq. 5% LiCl-solution, once with water and once with brine. The combined organic layers were dried over sodium sulfate, filtered and concentrated in vacuo. The crude product was adsorbed on ISOLUTE HM-N and purified by flash chromatography (silica gel, 25 g, gradient 0% to 50% ethyl acetate in heptane) to afford the title compound (603 mg, 84% yield) as a light yellow oil. LCMS: m/z 339.2 [M+H]⁺, ESI pos.

### Step B: 1-(6-Chloropyridazin-3-yl)-2,3,3a,4,5,6,7,7a-octahydropyrrolo[2,3-c]pyridine hydrochloride

To a solution of tert-butyl 1-(6-chloropyridazin-3-yl)-3,3a,4,5,7,7a-hexahydro-2H-pyrrolo[2,3-c]pyridine-6-carboxylate (Example 6, step A) (596 mg, 1.67 mmol, 1.00 eq) in dichloromethane (8.0 mL) was added dropwise 4 M HCl in dioxane (5.04 g, 4.2 mL, 16.8 mmol, 10.05 eq). The reaction mixture was stirred at room temperature for 16 hours. The reaction mixture was concentrated in vacuo to afford the title compound (665 mg, 94.% yield, 65% purity) as a light yellow foam, which was used without further purification. LCMS: m/z 239.1 [M+H]⁺, ESI pos.

### Step C: 1-(6-Chloropyridazin-3-yl)-6-ethyl-3,3a,4,5,7,7a-hexahydro-2H-pyrrolo[2,3-c]pyridine

To a suspension of 1-(6-chloropyridazin-3-yl)-2,3,3a,4,5,6,7,7a-octahydropyrrolo[2,3-c]pyridine hydrochloride (Example 6, step B) (665 mg, 1.57 mmol, 1.00 eq, 65% purity) in dichloromethane (10 mL) was added acetaldehyde (172 mg, 0.22 mL, 3.90 mmol, 2.48 eq) followed by sodium acetate (260 mg, 3.17 mmol, 2.02 eq) under ice-bath cooling. Sodium triacetoxy borohydride (502 mg. 2.37 mmol, 1.51 eq) was added in three portions at 0° C. The reaction mixture was stirred at 0° C. for 30 minutes and at room temperature for 1 hour. The reaction mixture was carefully quenched with saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in dichloromethane) to afford the title compound (262 mg, 59% yield) as a brown solid. LCMS: m/z 267.2 [M+H]⁺, ESI pos.

### Step D: 4-[16-(6-Ethyl-3.3a,4,5,7,7a-hexahydro-2H-pyrrolo[2,3-c]pyridin-1-yl)pyridazin-3-yl]-3-hydroxy-5-methyl-benzonitrile

A mixture of 1-(6-chloropyridazin-3-yl)-6-ethyl-3,3a,4,5,7,7a-hexahydro-2H-pyrrolo[2,3-c]pyridine (Example 6, step C) (160 mg, 0.57 mmol, 1.00 eq), (4-cyano-2-hydroxy-6-methyl-phenyl)boronic acid (172 mg, 0.97 mmol, 1.71 eq, Intermediate 2E), cesium carbonate (557 mg, 1.71 mmol, 3.00 eq) and XPhos Pd G3 (72 mg, 0.09 mmol, 0.15 eq) in 1,4-dioxane (3.6 mL) and water (0.90 mL) was flushed with argon and stirred at 100° C. for 4 hours and at room temperature for 16 hours. The reaction mixture was extracted with ethyl acetate and half-saturated aq. NH4Cl-solution. The aqueous layer was back extracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (Si-amine, 12 g, gradient 0% to 10% methanol in ethyl acetate). All fractions containing product were combined and concentrated in vacuo. The residue was repurified by flash chromatography (silica gel, 12 g, gradient 0% to 50% (dichloromethane:methanol:NH₄OH 9:1:0.05) in dichloromethane) to afford the title compound (167 mg, 77% yield) as a light yellow foam. LCMS: m/z 364.3 [M+H]⁺, ESI pos.

### Step E: 4-[6-[(3aS,7aR)-6-Ethyl-3.3a,4.5.7.7a-hexahydro-2H-pyrrolo[2,3-c]pyridin-1-yl]pyridazin-3-yl]-3-hydroxy-5-methyl-benzonitrile and 4-[6-[(3aR,7aS)-6-ethyl-3.3a,4,5,7,7a-hexahydro-2H-pyrrolo[2,3-c]pyridin-1-yl]pyridazin-3-yl]-3-hydroxy-5-methyl-benzonitrile

Chiral separation by SFC (column chiral IK, eluent B: 40% methanol+0.2% diethylamine) of 4-[6-(6-ethyl-3,3a,4,5,7,7a-hexahydro-2H-pyrrolo[2,3-c]pyridin-1-yl)pyridazin-3-yl]-3-hydroxy-5-methyl-benzonitrile (Example 6, step D) (164 mg, 0.43 mmol, 1.00 eq) to afford the two enantiomers Example 6A (first eluting, Rₜ=1.98 minutes) (76 mg, 46% yield) as a light brown foam: LCMS: m/z 364.2 [M+H]⁺, ESI pos and example 6B (second eluting, Rₜ=2.55 minutes) (78 mg, 48% yield) as a light brown foam: LCMS: m/z 364.3 [M+H]⁺, ESI pos.

### Example 7, 7A and 7B

### 3-Hydroxy-4-[4-methyl-6-(6-methyl-3,3a,4,5,7,7a-hexahydro-2H-pyrrolo[2,3-c]pyridin-1-yl)pyridazin-3-yl]benzonitrile, 4-[6-[(3aS,7aR)-6-methyl-3,3a,4,5,7,7a-hexahydro-2H-pyrrolo[2,3-c]pyridin-1-yl]-4-methyl-pyridazin-3-yl]-3-hydroxy-benzonitrile and 4-[6-[(3aR,7aS)-6-methyl-3,3a,4,5,7,7a-hexahydro-2H-pyrrolo[2,3-c]pyridin-1-yl]-4-methyl-pyridazin-3-yl]-3-hydroxy-benzonitrile

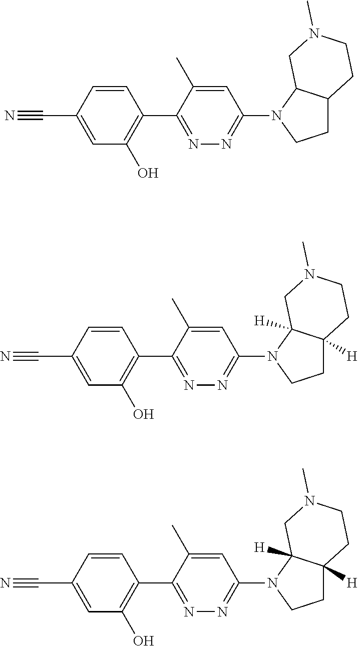 [SMILES: Cc1cc(N2CCC3CCN(C)CC32)nnc1-c1ccc(C#N)cc1O ; Cc1cc(N2CC[C@@H]3CCN(C)C[C@@H]32)nnc1-c1ccc(C#N)cc1O ; Cc1cc(N2CC[C@H]3CCN(C)C[C@H]32)nnc1-c1ccc(C#N)cc1O]

### Step A: tert-Butyl 1-(6-chloro-5-methyl-pyridazin-3-yl)-3.3a,4,5,7,7a-hexahydro-2H-pyrrolo[2,3-c]pyridine-6-carboxylate

3,6-Dichloro-4-methyl-pyridazine (200 mg, 1.23 mmol, 1.0 eq) was dissolved in NMP (2.0 mL) and 1,2,3,3a,4,5,7,7a-octahydropyrrolo[2,3-c]pyridine-6-carboxylic acid tert-butyl ester (277.69 mg, 1.23 mmol, 1.0 eq, CAS #1196147-27-9) and N,N-diisopropylethylamine (641.3 mg, 867 μL, 4.96 mmol, 4.0 eq) were added to the reaction mixture and it was stirred at 130° C. for five hours. After cooling to r.t., the reaction mixture was extracted with ethyl acetate (10 mL) and aq. LiCl-solution (10%. 2.0 mL). The organic layer was washed two times with aq. LiCl-solution (10%, 2.0 mL), once with water (5.0 mL) and once with brine (5.0 mL). The aqueous layers were back-extracted with ethyl acetate (10 mL). The combined organic layers were dried over sodium sulfate, filtered and concentrated in vacuo. The crude product was purified by flash chromatography on silica gel, (gradient 0% to 50% EtOAc in heptane) and repurified by SFC (coloumn: achiral Torus2Pic, 12 nm, 5 μm, 250×20 mm, 10% MeOH) to yield the title compound (197.4 mg, 41% yield) as light yellow solid and tert-butyl 1-(6-chloro-4-methyl-pyridazin-3-yl)-3,3a, 4,5,7,7a-hexahydro-2H-pyrrolo[2,3-c]pyridine-6-carboxylate (58 mg, 13%) as light yellow solid. LCMS m/z: 353.2 [M+H]⁺, ESI pos.

### Step B: 1-(6-Chloro-5-methyl-pyridazin-3-yl)-2,3,3a,4,5,6,7,7a-octahydropyrrolo[2,3-c]pyridine; hydrogen chloride

Aforementioned tert-butyl 1-(6-chloro-5-methyl-pyridazin-3-yl)-3,3a,4,5,7,7a-hexahydro-2H-pyrrolo[2,3-c]pyridine-6-carboxylate (197 mg. 0.5 mmol, 1.0 eq) was dissolved in dichloromethane (1.6 mL) and methanol (0.8 mL). Then, 4 M HCl in dioxane (1.2 mL, 4.78 mmol, 9.0 eq) was added dropwise to the reaction mixture and it was stirred at r.t. for two hours. The reaction mixture was concentrated in vacuo afford the title compound (180 mg. 88% yield) as light yellow solid. LCMS m/z: 253.2 [M+H]⁺, ESI pos.

### Step C: 1-(6-Chloro-5-methyl-pyridazin-3-yl)-6-methyl-3,3a,4,5,7,7a-hexahydro-2H-pyrrolo[2,3-c]pyridine

Aforementioned 1-(6-chloro-5-methyl-pyridazin-3-yl)-2,3,3a,4,5,6,7,7a-octahydropyrrolo[2,3-c]pyridine: hydrogen chloride (200 mg. 691.6 μmol, 1.0 eq) was dissolved in 1,2-dichloroethane (6.6 mL) and triethylamine (107.8 mg, 148 μL, 1.06 mmol, 1.54 eq) was added. After stirring for 5 minutes, formaldehyde, (37% aq. solution, 125.8 mg. 115.4 μL, 1.6 mmol, 2.2 eq) was added followed by portion-wise addition of sodium triacetoxy borohydride (586.3 mg, 2.77 mmol, 4.0 eq). The reaction mixture was stirred at room temperature for one hour. The reaction mixture was carefully quenched with saturated aq. NaHCO₃-solution and extracted five times with DCM (+2% MeOH). The combined organic layers were dried over sodium sulfate, filtered and concentrated in vacuo. The crude product was purified by flash chromatography on silica gel (gradient 0% to 5% methanol in dichloromethane) to afford the title compound (123 mg. 63% yield) as light brown solid. LCMS m/z: 267.2 [M+H]⁺, ESI pos.

### Step D: 3-Hydroxy-4-[4-methyl-6-(6-methyl-3.3a,4.5.7.7a-hexahydro-2H-pyrrolo[2,3-c]pyridin-1-yl)pyridazin-3-yl]benzonitrile

A mixture of aforementioned 1-(6-chloro-5-methyl-pyridazin-3-yl)-6-methyl-3,3a,4,5,7,7a-hexahydro-2H-pyrrolo[2,3-c]pyridine (50 mg, 187 μmol, 1.0 eq), commercially available (4-cyano-2-hydroxy-phenyl)boronic acid (49.5 mg. 304 μmol, 1.6 eq: no CAS #), potassium carbonate (119 mg. 860 μmol, 4.6 eq) and 1,1′-bis(diphenylphosphino)ferrocene-palladium(ii) dichloride dichloromethane complex (23.1 mg. 28.3 μmol, 0.15 eq) in 1,4-dioxane (1.19 mL) and water (0.59 mL) was flushed with argon and stirred at 95° C. overnight. Afterwards, (4-cyano-2-hydroxy-phenyl)boronic acid (49.53 mg, 304 μmol, 1.6 eq) and 1,1′-bis(diphenylphosphino)ferrocene-palladium(ii) dichloride dichloromethane complex (23.1 mg, 28.3 μmol, 0.15 eq) was added to the reaction mixture and stirring continued at 95° C. for five hours. The reaction mixture was cooled to r.t. and then extracted with ethyl acetate and half-saturated NH₄Cl-solution. The aqueous layer was back-extracted with ethyl acetate. The organic layers were washed with water and brine. The combined organic layers were dried over sodium sulfate, filtered and concentrated in vacuo. The crude product was purified by flash chromatography on silica gel (gradient 0% to 10% methanol in dichloromethane) and re-purified by preparative HPLC (column: Gemini NX, 12 nm, 5 μm, 100×300 mm: gradient MeCN/water +0.1% TEA) to afford the title compound (8 mg, 12% yield) as white solid. LCMS m/z: 348.2 [M−H]⁻, ESI neg.

### Step E: 4-[6-[(3aS,7aR)-6-Methyl-3.3a,4,5,7,7a-hexahydro-2H-pyrrolo[2,3-c]pyridin-1-yl]-4-methyl-pyridazin-3-yl]-3-hydroxy-benzonitrile and 4-[6-[(3aR,7aS)-6-methyl-3.3a,4,5,7,7a-hexahydro-2H-pyrrolo[2,3-c]pyridin-1-yl]-4-methyl-pyridazin-3-yl]-3-hydroxy-benzonitrile

Aforementioned 3-hydroxy-4-[4-methyl-6-(6-methyl-3,3a,4,5,7,7a-hexahydro-2H-pyrrolo[2,3-c]pyridin-1-yl)pyridazin-3-yl]benzonitrile (60 mg) was prepared as described above. After chiral separation via SFC (coloumn: chiral AD-H, 5 μm, 250×20 mm, 20% MeOH+0.2% DEA) compound 7A (first eluting, Rₜ=2.16 min: 20.6 mg, 8% yield) was isolated as a light yellow solid and compound 7B (second eluting, Rₜ=2.16 min: 11.4 mg, 4% yield) as a light yellow solid. LCMS m/z: 348.2 [M−H]⁻, ESI neg.

### Example 8

### 4-[4-Ethyl-6-[[(3R)-1-ethyl-3-piperidyl]amino]pyridazin-3-yl]-3-hydroxy-benzonitrile

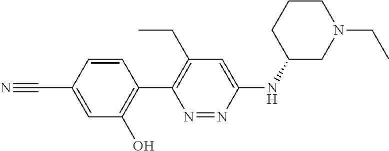 [SMILES: CCc1cc(N[C@@H]2CCCN(CC)C2)nnc1-c1ccc(C#N)cc1O]

Step A: tert-Butyl (3R)-3-[(6-chloro-5-ethyl-pyridazin-3-yl)amino]piperidine-1-carboxylate In a sealed tube, a neat mixture of (3R)-3-aminopiperidine-1-carboxylic acid tert-butyl ester (4.53 g, 22.6 mmol, 2.0 eq, CAS #188111-79-7) and 3,6-dichloro-4-ethyl-pyridazine (2 g, 11.3 mmol, 1.0 eq, CAS #10728-54-6) in N,N′-diisopropylethylamine (3.65 g, 4.93 mL, 28.2 mmol, 2.5 eq) was sealed and stirred at 130° C. (preheated oilbath) overnight. The viscous-brown reaction mixture was poured warm (˜50° C.) on water (˜100 ml) and ethyl acetate (˜50 ml) was used to transfer the oily mixture. The brown reaction solution was stirred for 10 minutes, transferred into a separating funnel and extracted twice with ethyl acetate (2×˜100 ml). The organic layers were washed with water (˜50 ml) and brine (˜50 ml). The combined organic extracts were dried over sodium sulfate, filtered and concentrated in vacuo. The brown crude product was purified by flash chromatography on silica gel (0%-40% ethyl acetate in heptane) to afford the title compound (1.32 g. 34% yield) as light yellow foam. LCMS m/z: 341.1 ([{35Cl}M+H]⁺), 341.1 ([{37C₁}M+H]⁺), ESI pos. As a second peak, (3R)-3-[(6-chloro-4-ethyl-pyridazin-3-yl)amino]piperidine-1-carboxylic acid tert-butyl ester (545 mg, 13% yield) was isolated as light yellow foam. LCMS: m/z 341.1 ([{35Cl}M+H]⁺), 341.1 ([{37C1}M+H]⁺), ESI pos.

### Step B: 6-Chloro-5-ethyl-N-[(3R)-3-piperidyl]pyridazin-3-amine; hydrogen chloride

To a solution of aforementioned tert-butyl (3R)-3-[(6-chloro-5-ethyl-pyridazin-3-yl)amino]piperidine-1-carboxylate (943 mg. 2.77 mmol, 1.0 eq) in dichloromethane (10 mL) and methanol (5 mL) was added dropwise at ambient temperature 4 M HCl in 1,4-dioxane (8.3 g, 6.92 mL, 27.67 mmol, 10 eq). The reaction mixture was stirred at 23° C. for 16 hours. After removal of the solvents under reduced pressure, the title compound was obtained (786 mg. 97% yield) as light yellow solid. LCMS: m/z 241.1 ([{35C1}M+H]⁺), 243.1 ([{37C1}M+H]⁺), ESI pos.

### Step C: 6-Chloro-5-ethyl-N-[(3R)-1-ethyl-3-piperidyl]pyridazin-3-amine

To a suspension of aforementioned 6-chloro-5-ethyl-N-[(3R)-3-piperidyl]pyridazin-3-amine: hydrogen chloride (400 mg, 1.44 mmol, 1.0 eq) in dichloromethane, extra dry (15 mL) was added acetaldehyde (158.9 mg. 204 μL, 3.6 mmol, 2.5 eq) followed by the addition of sodium acetate (296 mg. 3.61 mmol, 2.5 eq) under ice-bath cooling. Afterwards, sodium triacetoxy borohydride (562.6 mg. 2.65 mmol, 1.8 eq) was added at 0° C. and stirring was continued for 5 min and then at room temperature for 3 hours. The reaction mixture was carefully basified with NaHCO₃-solution (25 mL) and then extracted with dichloromethane (3×60 mL). The combined organic layers were dried over sodium sulfate, filtered and concentrated in vacuo. The crude product was purified by flash chromatography on silica gel (gradient 0% to 100%: dichloromethane:methanol:NH₄OH 110:10:1) in dichloromethane) to afford the title compound (204 mg, 53% yield) as light brown oil. MS: m/z 269.1 ([{35Cl}M+H]⁺), 271.1 ([{37C1}M+H]⁺), ESI pos.

### Step D: 4-[4-Ethyl-6-[[(3R)-1-ethyl-3-piperidyl]amino]pyridazin-3-yl]-3-hydroxy-benzonitrile

To a mixture of aforementioned (6-chloro-5-ethyl-pyridazin-3-yl)-[(3R)-1-ethyl-3-piperidyl]amine (134 mg. 499 μmol, 1.0 eq), commercially available (4-cyano-2-hydroxy-phenyl)boronic acid (118.5 mg. 698 μmol, 1.4 eq, no CAS #) and cesium carbonate (487.3 mg, 1.5 mmol, 3.0 eq) in 1,4-dioxane (4 mL) and water (1 mL) was added under argon XPhos Pd G3 (63.3 mg, 74.78 μmol, 0.15 eq, CAS #1445085-55-1). The reaction mixture was stirred in a sealed tube at 100° C. for 4 hours. The reaction mixture was extracted with ethyl acetate (2×40 mL) and half-saturated NH₄Cl-solution (40 mL). The organic layers were washed with water (40 mL) and brine (40 mL). The combined organic layers were dried over sodium sulfate, filtered and concentrated in vacuo. The crude product was purified by flash chromatography on silica gel (gradient 0% to 100% (dichloromethane:methanol:NH₄OH 110:10:1) in dichloromethane) followed by preparative HPLC to afford the title compound (44 mg, 25% yield) as white amorph freeze-dried solid. LCMS m/z: 352.3 [M+H]⁺, ESI pos.

### Example A′

A compound of formula Ib can be used in a manner known per se as the active ingredient for the production of tablets of the following composition:

### Per Tablet

| | |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B′

A compound of formula Ib can be used in a manner known per se as the active ingredient for the production of capsules of the following composition:

### Per Capsule

| | |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

### Example A

A compound of formula I can be used in a manner known per se as the active ingredient for the production of tablets of the following composition:

### Per Tablet

| | |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula I can be used in a manner known per se as the active ingredient for the production of capsules of the following composition:

### Per Capsule

| | |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. A compound of formula Ib 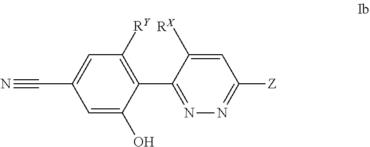
wherein
R^{X} is selected from H, alkyl and haloalkyl and R^{Y} is selected from H, alkyl, alkoxyalkyl and halo, provided that if R^{X} is H then R^{Y} is not H;
Z is selected from systems R, S, and T 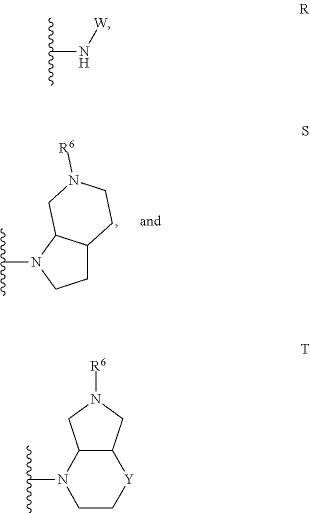
wherein systems S and T can be further substituted by OH, halo, alkyl or cyano;
R⁶ is H or alkyl;
Y is CH₂, O or NR⁷;
R⁷ is H or alkyl;
W is a substituted 4-6-membered-cycloalkyl ring or a substituted heterocycle of ring system A, wherein substituted cycloalkyl is substituted with 1 or 2 substituents selected from OH, halo, and alkyl, and ring system A is 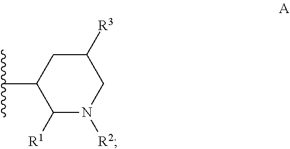
R¹ is H and R² is alkyl, or R¹ and R², and the atoms to which they are bonded, together form a 5-member ring optionally substituted with OH or halo;
R³ is H, OH or halo;
and pharmaceutically acceptable salts thereof.

2. A compound according to claim 1, wherein R^{X} is H, alkyl or haloalkyl.

3. A compound according to claim 1, wherein R^{X} is H or alkyl.

4. A compound according to claim 1, wherein R^{Y} is H or alkyl.

5. A compound according to any of claim 1, wherein Z is system R or system S.

6. A compound according to claim 1, wherein Z is system S.

7. A compound according to claim 1, wherein R⁶ is alkyl.

8. A compound according to claim 1, wherein W is a 4-membered cycloalkyl ring substituted with alkyl and OH, or W is a substituted heterocycle of ring system A wherein R¹ is H, R² is alkyl and R³ is H;

9. A compound according to claim 1, wherein
R^{X} is selected from H, alkyl and haloalkyl and R^{Y} is selected from H and alkyl, provided that if R^{X} is H then R^{Y} is not H;
Z is selected from systems R and S 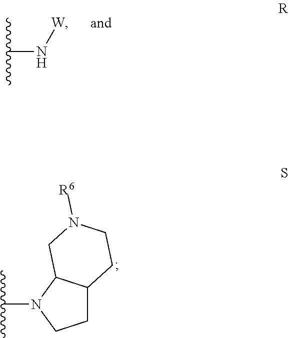
R⁶ is alkyl;
W is a substituted 4 membered-cycloalkyl ring substituted with alkyl and OH, or W is a substituted heterocycle of ring system A, 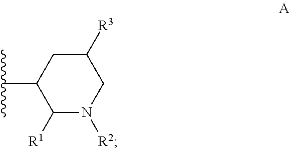
R¹ is H and R² is alkyl;
R³ is H;
and pharmaceutically acceptable salts thereof.

10. A compound according to claim 1, wherein
R^{X} is selected from H and alkyl and R^{Y} is selected from H and alkyl, provided that if R^{X} is H then R^{Y} is not H;
Z is system S 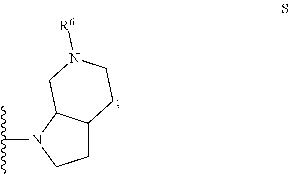
R⁶ is alkyl;
and pharmaceutically acceptable salts thereof.

11. A compound according to claim 1, wherein the compound is selected from
4-[6-[[(3R)-1-Ethyl-3-piperidyl]amino]-4-methyl-pyridazin-3-yl]-3-hydroxy-benzonitrile formic acid; and
4-[6-[[(3R)-1-Ethyl-3-piperidyl]amino]-4-methyl-pyridazin-3-yl]-3-hydroxy-benzonitrile;
and pharmaceutically acceptable salts thereof.

12. A compound according to claim 1, wherein the compound is selected from
4-[6-[[(3R)-1-Ethyl-3-piperidyl]amino]pyridazin-3-yl]-3-hydroxy-5-methyl-benzonitrile;
4-[6-[[(3R)-1-Ethyl-3-piperidyl]amino]-4-(trifluoromethyl)pyridazin-3-yl]-3-hydroxy-benzonitrile;
3-Hydroxy-4-[6-[(3-hydroxy-3-methyl-cyclobutyl)amino]-4-methyl-pyridazin-3-yl]benzonitrile;
4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aS,7aR)-6-Methyl-3,3a,4,5,7,7a-hexahydro-2H-pyrrolo[2,3-c]pyridin-1-yl]pyridazin-3-yl]-3-hydroxy-5-methyl-benzonitrile;
and pharmaceutically acceptable salts thereof.

13. A compound according to claim 1, wherein the compound is selected from
4-[6-[(3aS,7aR)-6-Ethyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aR,7aS)-6-Ethyl-3,3a,4,5,7,7a-hexahydro-2H-pyrrolo[2,3-c]pyridin-1-yl]pyridazin-3-yl]-3-hydroxy-5-methyl-benzonitrile;
3-Hydroxy-4-[4-methyl-6-(6-methyl-3,3a,4,5,7,7a-hexahydro-2H-pyrrolo[2,3-c]pyridin-1-yl)pyridazin-3-yl]benzonitrile;
4-[6-[(3aS,7aR)-6-Methyl-3,3a,4,5,7,7a-hexahydro-2H-pyrrolo[2,3-c]pyridin-1-yl]-4-methyl-pyridazin-3-yl]-3-hydroxy-benzonitrile;
4-[6-[(3aR,7aS)-6-Methyl-3,3a,4,5,7,7a-hexahydro-2H-pyrrolo[2,3-c]pyridin-1-yl]-4-methyl-pyridazin-3-yl]-3-hydroxy-benzonitrile;
4-[4-Ethyl-6-[[(3R)-1-ethyl-3-piperidyl]amino]pyridazin-3-yl]-3-hydroxy-benzonitrile;
and pharmaceutically acceptable salts thereof.

14. A compound according to claim 1, wherein the compound is selected from
4-[6-[(3aR,7aS)-6-Methyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aS,7aR)-6-methyl-3,3a,4,5,7,7a-hexahydro-2H-pyrrolo[2,3-c]pyridin-1-yl]pyridazin-3-yl]-3-hydroxy-5-methyl-benzonitrile;
and pharmaceutically acceptable salts thereof.

15. A compound according to claim 1, wherein the compound is selected from
4-[6-[(3aS,7aR)-6-Ethyl-3,3a,4,5,7,7a-hexahydro-2H-pyrrolo[2,3-c]pyridin-1-yl]pyridazin-3-yl]-3-hydroxy-5-methyl-benzonitrile;
4-[6-[(3aR,7aS)-6-Ethyl-3,3a,4,5,7,7a-hexahydro-2H-pyrrolo[2,3-c]pyridin-1-yl]pyridazin-3-yl]-3-hydroxy-5-methyl-benzonitrile;
3-Hydroxy-4-[4-methyl-6-(6-methyl-3,3a,4,5,7,7a-hexahydro-2H-pyrrolo[2,3-c]pyridin-1-yl)pyridazin-3-yl]benzonitrile;
4-[6-[(3aS,7aR)-6-Methyl-3,3a,4,5,7,7a-hexahydro-2H-pyrrolo[2,3-c]pyridin-1-yl]-4-methyl-pyridazin-3-yl]-3-hydroxy-benzonitrile;
4-[6-[(3aR,7aS)-6-Methyl-3,3a,4,5,7,7a-hexahydro-2H-pyrrolo[2,3-c]pyridin-1-yl]-4-methyl-pyridazin-3-yl]-3-hydroxy-benzonitrile;
and pharmaceutically acceptable salts thereof.

16. (canceled)

17. (canceled)

18. A pharmaceutical composition comprising a compound according to claim 1 and a therapeutically inert carrier.

19-23. (canceled)

24. A method of inhibiting NLRP3, which method comprises administering an effective amount of a compound as claimed in claim 1 to inhibit NLRP3.

25. A method for the treatment or prophylaxis of a disease, disorder or condition, which method comprises administering an effective amount of a compound according to claim 1, wherein the disease, disorder or condition is selected from Asthma or COPD.

26. A method for the treatment or prophylaxis of a disease, disorder or condition, which method comprises administering an effective amount of a compound according to claim 1, wherein the disease, disorder or condition is selected from Parkinson's Disease or Alzheimer's Disease.

27. (canceled)
